(19) United States
(12) Patent Application Publication
(10) Pub. No.: US 2021/0350247 A1
(43) Pub. Date: Nov. 11, 2021

(54) HYBRID DECISION TREE MACHINE LEARNING SYSTEMS AND METHODS

(71) Applicant: Vision Source L.P., Kingwood, TX (US)

(72) Inventors: Jessica Stoffel, St. Louis, MO (US); Kurt Goolsbee, Spring, TX (US); Barry Turney, Coldspring, TX (US)

(73) Assignee: Vision Source L.P., Kingwood, TX (US)

(21) Appl. No.: 17/314,278

(22) Filed: May 7, 2021

Related U.S. Application Data
(60) Provisional application No. 63/021,769, filed on May 8, 2020.

(51) Int. Cl. G06N 5/00; G16H 20/00; G16H 10/60

(52) U.S. Cl. CPC G06N 5/003; G16H 10/60; G16H 20/00

(57) ABSTRACT

Disclosed are implementations of systems and methods for generation and execution of hybrid decision trees for machine learning systems and processing environments. The systems include a data processing system for a computer memory, comprising means for configuring a memory device according to a hybrid decision tree, said hybrid decision tree including a plurality of nodes, each node comprising at least one directional associative link to or from another node, each node associated with a function performed on an input variable, wherein each directional associative link is agnostic to a hierarchical layer of the associated nodes in the hybrid decision tree; and means for executing functions associated with each node of the hybrid decision tree according to the corresponding directional associative links.

### RELATED APPLICATIONS

The present disclosure claims the benefit of and priority to U.S. Provisional Patent Application No. 63/021,769, entitled “System and Method for Treatment Package Identification and Delivery,” filed May 8, 2020, the entirety of which is incorporated by reference herein.

### FIELD

The present disclosure relates generally to a system and method for identifying and delivering treatment packages.

### BACKGROUND

The statements in this section merely provide background information related to the present disclosure and may not constitute prior art.

Some implementations of machine learning systems use decision trees, or multi-level hierarchical structures with nodes corresponding to questions or decisions, and leaf nodes corresponding to outcome values. In many implementations, decision trees may be highly intuitive and generate justifiable classifications or decisions of input data. However, in many implementations, machine learning decision trees may be restricted in functionality due to their inherent structure.

### SUMMARY

To address the above-discussed and other problems, the present disclosure provides implementations of systems and methods for hybrid independent-dependent decision trees for machine learning systems and/or computer automation systems. These implementations are not limited to a static hierarchy of standard single-layer parent-child relationships, but rather utilize an abstracted relational structure that enables a mixture of independent and dependent nodes, providing greater flexibility and functionality than purely independent-node decision trees, and reduced structure sizes and faster processing relative to purely dependent-node decision trees. The implementations of hybrid independent-dependent decision trees discussed herein, along with the systems and methods for generation and use, thus provide a technological improvement to the field of data processing.

### BRIEF DESCRIPTION OF THE DRAWINGS

For a more complete understanding of various embodiments of the present disclosure and the advantages thereof, reference is now made to the following brief description, taken in connection with the accompanying drawings and detailed description, wherein like reference numerals represent like parts, and in which:

FIG. 1A illustrates an embodiment of an independent decision tree;

FIG. 1B illustrates an embodiment of a dependent decision tree;

FIG. 1C illustrates an embodiment of a hybrid decision tree;

FIG. 1D illustrates a system for creation and use of hybrid decision trees, in accordance with various embodiments;

FIG. 2A illustrates a system for treatment package identification and delivery, in accordance with various embodiments;

FIG. 2B illustrates a method of treatment package identification and delivery, in accordance with various embodiments;

FIG. 3 illustrates a first set of screen display objects represented on a human-readable interface in accordance with example systems and methods of treatment package identification and delivery disclosed herein;

FIG. 4 illustrates a second set of screen display objects represented on a human-readable interface in accordance with example systems and methods of treatment package identification and delivery disclosed herein;

FIG. 5 illustrates a sixth set of screen display objects represented on a human-readable interface in accordance with example systems and methods of treatment package identification and delivery disclosed herein; and

FIG. 6 illustrates a seventh set of screen display objects represented on a human-readable interface in accordance with example systems and methods of treatment package identification and delivery disclosed herein.

### DETAILED DESCRIPTION

The present disclosure is generally described in detail with reference to embodiments illustrated in the drawings. However, other embodiments may be used and/or other changes may be made without departing from the spirit or scope of the present disclosure. The illustrative embodiments described in the detailed description are not meant to be limiting of the subject matter presented herein.

Reference will now be made to the exemplary embodiments illustrated in the drawings, and specific language will be used to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

Some implementations of machine learning systems use decision trees, or multi-level hierarchical structures with nodes corresponding to questions or decisions, and leaf nodes corresponding to outcome values. In many implementations, decision trees may be highly intuitive and generate justifiable classifications or decisions of input data. However, in many implementations, machine learning decision trees may be restricted in functionality due to their inherent structure.

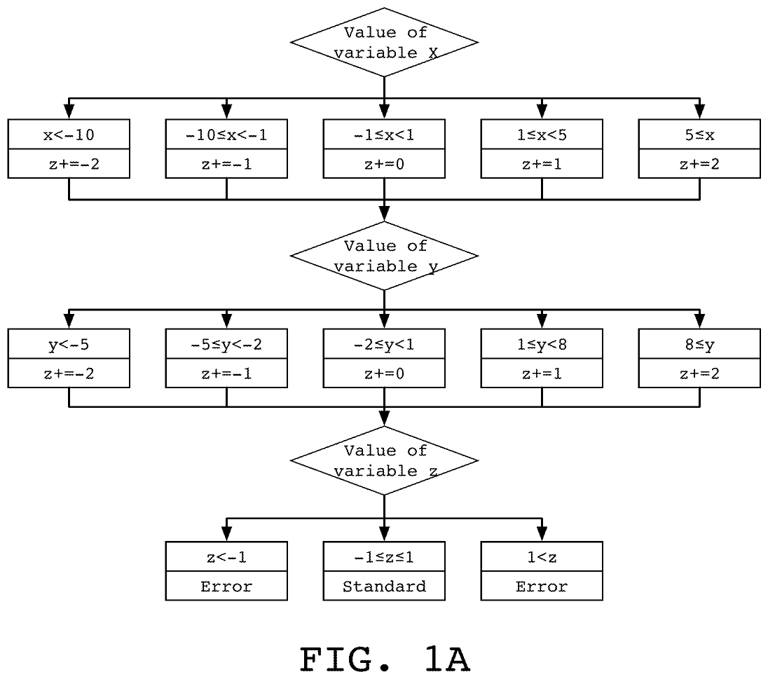

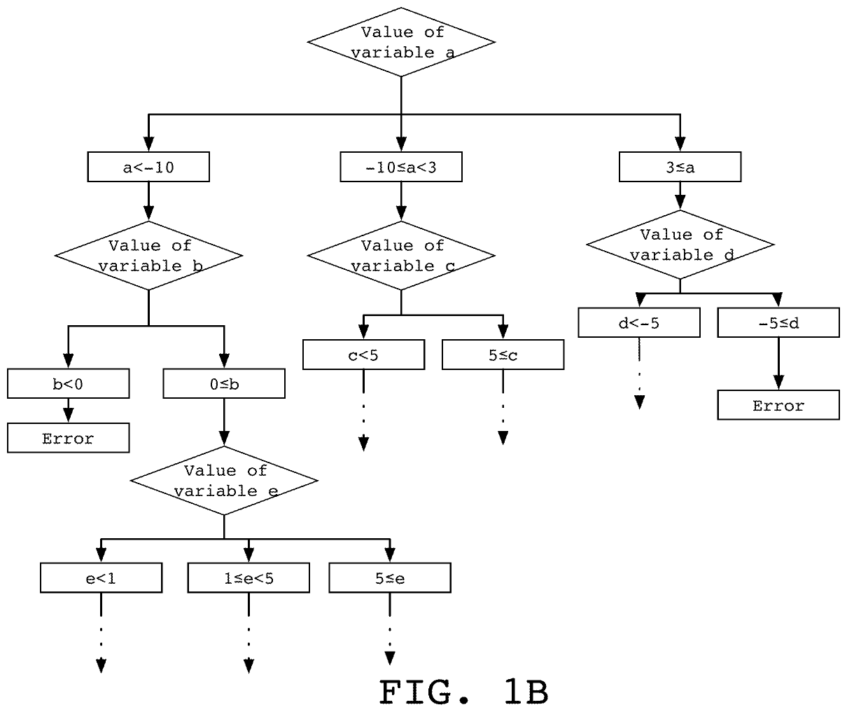

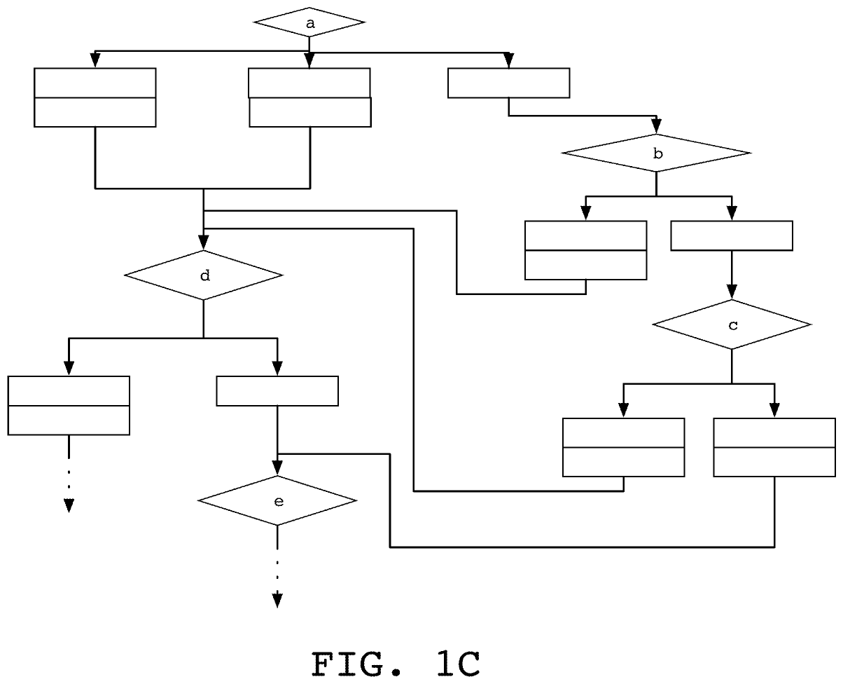

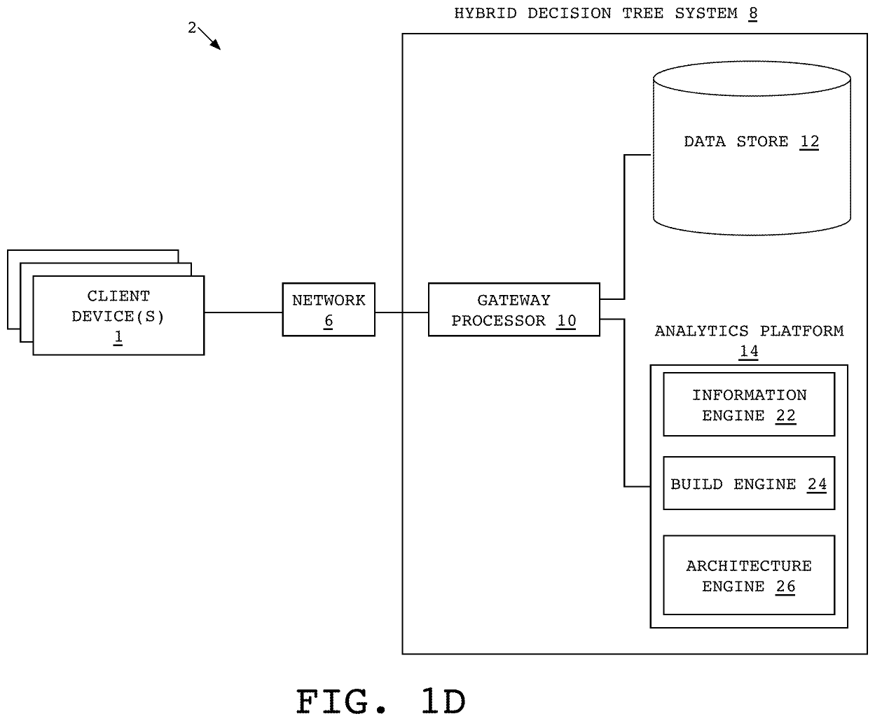

For example, FIG. 1A illustrates an embodiment of an independent decision tree, in which each decision node (illustrated as a diamond) is reached and applied, regardless of the outcome of prior decisions at higher layers (e.g. parent nodes) of the tree. For example, in the illustrated example, the variable z is set to a first value based on an input variable x, and then the variable z is adjusted based on a second input variable y (in the illustrated embodiment, the stacked rectangles provide a conditional filter applied to the input variable on top, and a resulting action to be applied if the condition matches the input on bottom). The final value of variable z is then compared to a final set of conditions to result in classifications in the leaf nodes at the bottom. Independent decision trees may be relatively compact and quick to traverse in a linear fashion, but may be inflexible. For example, in many implementations, a first variable may be dependent on a second variable such that the first variable may be considered for aggregation into the output if and only if the second variable has a given value. If the second variable does not have the given value, processing the decision tree by applying this irrelevant dependent node wastes resources.

By contrast, FIG. 1B illustrates an embodiment of a dependent decision tree, in which each node is associated with a single parent node and associated with a plurality of child nodes (which may include leaf or terminal nodes). Unlike the independent decision tree of FIG. 1A, the dependent decision tree provides greater flexibility by ignoring irrelevant comparisons if they are not triggered by the result of a higher layer node and comparison (e.g. the right-most decision based on the value of variable d is only reached if the first comparison determines that variable a is greater than 3). However, even simple dependent decision trees may grow very wide, with many, many nodes at each new child layer. In particular, the dependent trees must be carefully constructed such that any dependent variables are placed at higher layers in the hierarchy, to avoid redundancy. For example, in the tree illustrated in FIG. 1B, a value of variable e is determined if the value of variable b is greater than 0 and the value of variable a is less than −10; however, if variable e also needs to be determined if variable d is less than −5, the variable e node would need to be duplicated with a second variable e′ node dependent from the variable d node. Conversely, the tree could be restructured with variable e at a higher layer of the hierarchy above variable a, such that the single node is reached regardless of the values of variable a, b, and d (though this may waste resources if the value of a is between −10 and 3, such that neither variable b nor d are considered). Accordingly, the flexibility provided by dependent decision trees comes with significant complexity and difficulty of construction of the tree, and with frequent redundancies resulting in wasted memory or processing resources.

Instead, the present disclosure provides implementations of systems and methods for hybrid independent-dependent decision trees for machine learning systems and/or computer automation systems. These implementations are not limited to a static hierarchy of standard single-layer parent-child relationships, but rather utilize an abstracted relational structure that enables a mixture of independent and dependent nodes, providing greater flexibility and functionality than purely independent-node decision trees, and reduced structure sizes and faster processing relative to purely dependent-node decision trees. The implementations of hybrid independent-dependent decision trees discussed herein, along with the systems and methods for generation and use, thus provide a technological improvement to the field of data processing.

FIG. 1C illustrates an embodiment of a hybrid decision tree (with conditions and results, represented by rectangular boxes) removed for clarity and simplicity of description). As shown, and unlike the independent tree of FIG. 1A and dependent tree of FIG. 1B, nodes in the hybrid tree of FIG. 1C may be freely interconnected with each other at any level of the hierarchy (and thus, the hierarchy itself may be considered to not have a set of discrete layers, but rather simply represent parent-child relationships between pairs of nodes but agnostic to generations or layers). Implementations of the hybrid tree provide both the flexibility of dependent trees with the narrower, less complex structure of independent trees, resulting in fewer resources required and more efficient and faster processing.

FIG. 1D illustrates a system 2 for creation and use of hybrid decision trees, in accordance with various embodiments. A hybrid decision tree computing system 8, which may comprise one or more computing devices including physical computing devices and virtual computing devices provided by one or more physical computing devices (e.g. cloud computing services) may communicate via one or more networks 6 with one or more client devices 1 (which may themselves comprise physical and/or virtual computing devices).

The system may include a gateway processor 10, sometimes referred to as a central processing unit, tensor processing unit, co-processor, core, or other processing circuitry, which may execute an analytics platform 14 on one or more hybrid trees stored in data store 12. Analytics platform 14, which may be embodied in software executed by processor 10 or may be implemented in hardware (e.g. an ASIC or FPGA circuit), may be configured to generate and/or execute the hybridized decision tree. For example, analytics platform may comprise an information engine 22 for ingestion of variable input data, conditionals, and/or output states from client devices 1. These data and/or conditionals may be provided in any suitable type and format, such as XML data, relational databases, flat files, arrays, or other data structures. Analytics platform 14 may comprise a build engine 24 for generating the hybrid decision tree structure from the input data, based on identified relationships between each condition and/or variable. For example, in some implementations, build engine 24 may generate directional associations between nodes based on identified parent-child relationships while agnostic to layer or generation within the tree, such that the resulting tree may be traversed along the directional associations. Analytics platform 14 may also comprise an architecture engine 26, which may be configured to execute the decision tree based on input variables received from one or more client devices 1. For example, in some implementations, architecture engine 26 may comprise an input form, parser, series of questions, or other such interface for receiving input data. Once received, the architecture engine may traverse the tree along directional associative links between parent-child nodes (regardless of layer) while applying conditionals and modifying output states (e.g. calculating intermediate scores, aggregating variables or selections, etc.) until reaching a leaf node and providing a final output (e.g. outputting states, variable values, or other data according to the leaf node configuration).

Accordingly, this disclosure is directed to implementations of systems and methods for hybrid independent-dependent decision trees for machine learning systems and/or computer automation systems. These implementations provide greater flexibility and functionality relative to purely independent-node decision trees, and reduced structure sizes and faster processing relative to purely dependent-node decision trees. In some aspects, the present disclosure describes implementations of a data processing system for a computer memory, comprising means for configuring a memory device according to a hybrid decision tree, said hybrid decision tree including: a plurality of nodes, each node comprising at least one directional associative link to or from another node, each node associated with a function performed on an input variable, wherein each directional associative link is agnostic to a hierarchical layer of the associated nodes in the hybrid decision tree; and means for executing functions associated with each node of the hybrid decision tree according to the corresponding directional associative links. In some implementations, a first node at a first layer of the hybrid decision tree comprises a link to a second node at the first layer of the hybrid decision tree and a third node at a different second layer of the hybrid decision tree.

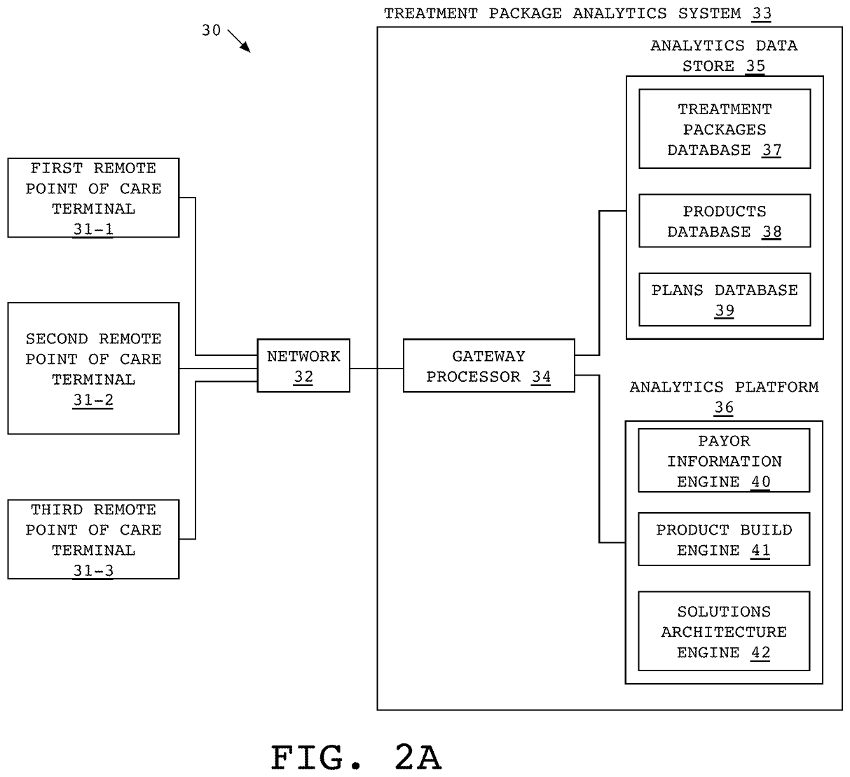

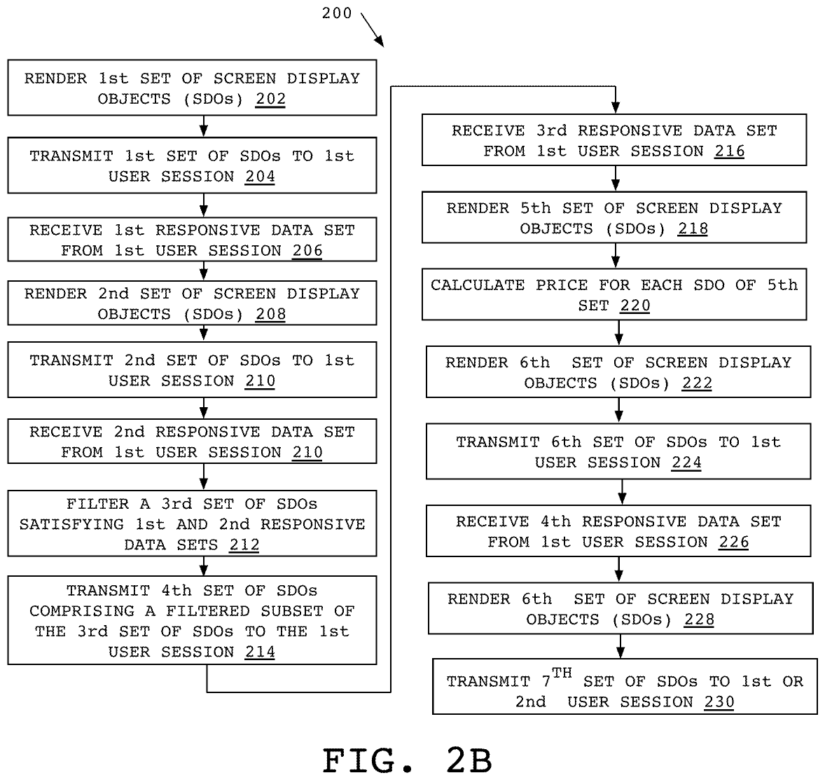

To better illustrate the usage of a hybrid independent-dependent decision tree, FIGS. 2A-6 and the accompanying description below provide an example of utilization of a hybrid decision tree for selection and configuration of a product or service, specifically systems and methods for helping patients select a treatment package. For example, a patient may be seeking to select an appropriate pair of glasses to treat a vision condition. Various different sets of glasses may be made up of various products such as frames, lenses, coatings, and the like. Thus, there are different treatment packages that can be assembled to effectively treat the vision condition. In addition, each patient may have different mechanisms for payment, for example, self-pay, a first insurance, a second insurance, and/or the like. These different arrangements for payment may be associated with different pricing and/or coverage of different products, leading to a preference for different treatment packages from one patient to the next. Suitable glasses for a patient may have myriad different configurations, different subsets of which are differently priced for different payment arrangements. Consequently, providing pricing data for treatment packages that are available to a specific patient becomes computationally intensive.

Yet furthermore, providing such pricing data may difficult or impossible in various treatment contexts. For example, a patient and a treatment provider may be remotely located from one another. Yet furthermore, one or both of a patient and treatment provider may be unable to install software directly on a local device. Thus, a distributed system provides a network-based solution for ready input of necessary data, completion of computationally intensive analysis, and display of output data to patients and treatment providers.

While the following description describes implementations of treatment package identification and delivery for illustrative purposes, as discussed above, the systems and methods discussed herein may be applied to any industry or environment utilizing hybrid machine learning decision trees.

With reference to FIG. 2A, an electronic system for treatment package identification and delivery 30 is provided. The electronic system for treatment package identification and delivery 30 may comprise various aspects as disclosed herein. For instance, one or more remote point of care terminals may be provided. A remote point of care terminal may be a computer, a tablet device, a smartphone device, or any other human-usable computing device as desired. FIG. 2A depicts any number ‘N’ of remote points of care terminals, such as a first remote point of care terminal 31-1, a second remote point of care terminal 31-2, and an third remote point of care terminal 31-3.

The remote point of care terminals may communicate with a treatment package analytics system 33 via a network 32. While in various embodiments, network 32 is the Internet, in further embodiments, network 32 may be a private network such as an intranet, or may be a virtual private network (VPN) running within a larger network, such as the Internet.

The treatment package analytics system 33 performs various aspects of the methods discussed herein, and may, via the network 32, provide output data for human viewing on one or more remote point of care terminal 31-1, 31-2, 31-3 and receive input data from human-machine interfaces of one or more remote point of care terminal 31-1, 31-2, 31-3. In various embodiments some inputs and/or outputs are provided at first remote point of care terminal 31-1 and some inputs and/or outputs are provided at second remote point of care terminal 31-2. FIG. 2A shows discrete modules representing aspects of the system 33. However, one may also recognize that one or more such modules may be distributed, such as by being one or more logical instances running on a distributed system. However, for ease of reference, the various modules will be discussed as discrete apparatuses making up the system 33.

A treatment package analytics system 33 may comprise a gateway processor 34. A gateway processor 34 may be an edge device sending data via network 32 and receiving data via network 32. Gateway processor 34 may control the flow of data among different modules of the system 33, and may limit access between network 32 (and other connected devices) with system 33. Gateway processor 34 may limit access among devices within system 33. In this manner, data may be secured and directed to appropriate destinations. Moreover, gateway processor 34 may include an electronic computer processor and a memory. In this manner, computations and decisions may be executed by the gateway processor 34.

A treatment package analytics system 33 may include an analytics data store 35. The analytics data store 35 comprises one or more database that contain the different treatment packages available, the different constituent products that make up each of the treatment packages, the different costs of the different products, the different insurance plans or other payment arrangements available, and the coverage of each insurance plan and/or payment arrangement for each different product or package. Though the analytics data store 35 is shown as a collection of discrete databases 37, 38, and 39, in various embodiments, the databases 37, 38, and 39 are representative depictions of different functional aspects of one or more shared database.

The analytics data store 35 may include a treatment packages database 37. The treatment packages database 37 contains the different combinations of products available for purchase as a bundle (e.g., a treatment package). Any package specific pricing information, such as rebates, or special pricing for combinations of products may be included.

The analytics data store 35 may include a products database 38. The products database 38 may include all the different products that make up different treatments, and information regarding each such product—for example price, compatibility with other products and the like.

The analytics data store 35 may include a plans database 39. The plans database 39 may include each available payment arrangement, such as the different possible insurance plans, and an indication of which product and/or which treatment package is covered. The plans database 39 further includes any special pricing information such as discounts for different products that may be associated with an available payment arrangement.

A treatment package analytics system 33 may include an analytics platform 36. The analytics platform 36 may include different engines configured to provide computational resources for different tasks. For example, a payor information engine 40, a product build engine 41 and a solution architecture engine 42 are provided. Each aspect of the analytics platform 36 will be discussed in greater detail in connection with the following explanation of one example method for treatment package identification and delivery using the provided electronic system for treatment package identification and delivery 2. Generally however, the payor information engine 40 comprises a logical portion of a processor structured and arranged to ingest data provided by a user at a remote point of care terminal 31-1, 31-2, 31-3 regarding a payor (e.g., data regarding a payment arrangement, medical condition of a patient, custom product selections made by a patient, and/or the like). The product build engine 41 may, based at least in part on the data regarding the payment arrangement, as well as on further data regarding a medical condition of a patient, generate proposed combinations of products effective at treating the medical condition. For example, the product build engine 41 may propose different treatment packages that reflect different pairs of glasses comprising different constituent products. Finally, a solution architecture engine 42 may ingest the proposed different combinations of products making up the proposed different treatment packages, as well as the data regarding the payment arrangement, and compute a price for each different treatment package for display to the patient at a remote point of care terminal based on whether that treatment package is available for that patient.

Having discussed aspects of the system 30 depicted in FIG. 2A, attention is now directed to FIG. 2B for a detailed discussion of an electronic method 200 of treatment package identification and delivery by an electronic system 30 for treatment package identification and delivery. Attention will also be given to FIG. 2A for further discussion of the different components of the electronic system for treatment package identification and delivery 30 that perform the disclosed method 200.

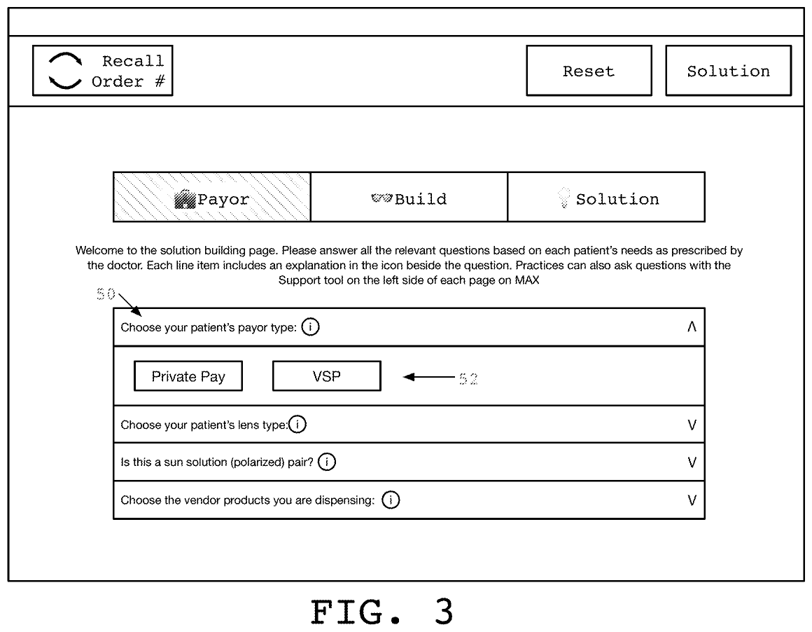

Referring to FIGS. 2A, 2B, and 3, a payor information engine 40 may render a first set of screen display objects 50 at least partially retrieved from a plans database 39 (block 202). These screen display objects represent different payment arrangements that a patient may choose. As used herein, “render” includes creating data representative of a user readable display for presentation to a human user. Render may include creating data for final provision to a user interface. Render may also include creating intermediate data, and/or incomplete data that is combined with other data prior to creating the final data stream provided to the user interface. As used herein, a “screen display object” includes a visual depiction corresponding to data. Thus, the payor information engine 40 assembles data that will allow a patient to choose which payment arrangement is desired.

A gateway processor 34 may transmit the first set of screen display objects to a first user session running on a first remote point of care terminal 31-1, wherein the first set of screen display objects 50 comprises a payor information solicitation (block 204). In various embodiments, the first set of screen display objects 50 comprises a sequence of clickable buttons 52 that are selectable to indicate which of a set of potential payment arrangements that a user possesses. This sequence may be termed the payor information solicitation. The different potential payment arrangements may correspond to different insurance plans and/or discount programs available to the user. In various embodiments, a first user session comprises a logged in session of a user via software on the first remote point of care terminal 31-1. In various instances, the user opens an internet browser and navigates to a website, logs in, and executes the first user session within an internet browser window of the first remote point of care terminal 31-1. The user may select one or more clickable button associated with one or more screen display object, thus choosing a payment arrangement from among various options.

The gateway processor 34 may receive, from the remote point of care terminal 31-1, via the network 32, a first responsive data set from the first user session, the first responsive data set comprising patient specific care plan information entered in response to the solicitation (block 206). The patient specific care plan information may comprise data representing a button click on a portion of the first set of screen display objects 50. The patient specific care plan information may comprise data representing a selection by a user of payor information associated with a particular choice of payor, such as an insurer or discount program provider. Thus, the first responsive data set represents the user's choice of a payor information in response to the payor information solicitation.

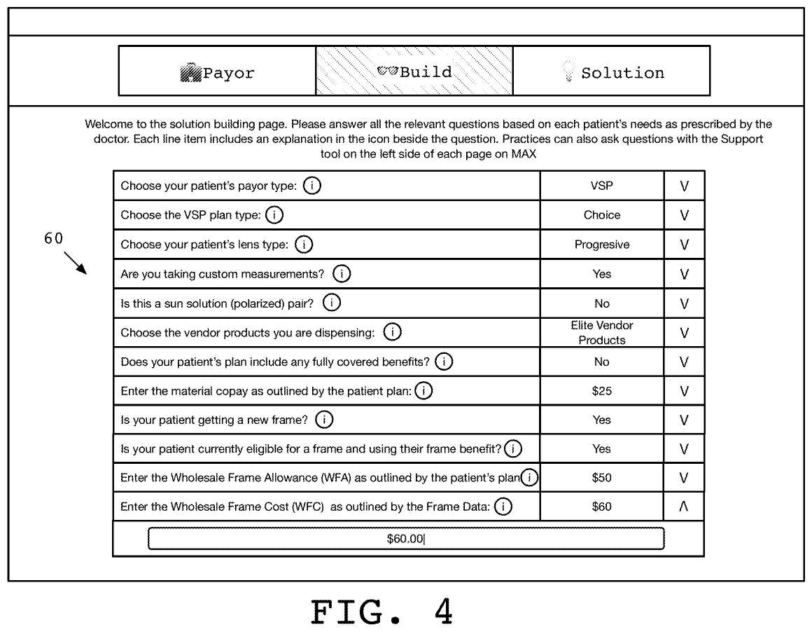

Referring to FIGS. 2A, 2B, and 4, the payor information engine 40 may render a second set of screen display objects 60 at least partially retrieved from a products database 38 (block 208). The gateway processor 34 may transmit the second set of screen display objects 60 to the first user session running on the first remote point of care terminal 31-1, wherein the second set comprises a medical data solicitation (block 210). A medical data solicitation may comprise data representing text, fields, buttons, and/or the like to prompt the entry by a user of the first remote point of care terminal 31-1 data reflecting medical conditions, measurements, and/or the like, the selection of which will gate the availability of different products from the products database 38 for the user.

The gateway processor 34 may receive, from the remote point of care terminal 31-1, via the network, a second responsive dataset from the first user session 31-1, the second responsive data set comprising patient specific treatment requirements entered in response to the medical data solicitation (block 210). Patient specific treatment requirements may comprise medical conditions, measurements, and/or the like, the selection of which will gate the availability of different products from the products database 38 for the user.

A product build engine 41 may perform a filtering operation. Specifically, the product build engine 41 may filter a third set of screen display objects comprising a set of customized care options stored in a products database 38 in response to the first responsive data set and the second responsive data set to create a first filtered subset of the customized care options that satisfy both the first responsive data set and the second responsive data set (block 212). Customized care options may comprise available variations in combinations of products effective at treating a medical condition of a patient. For instance, different vision conditions may require relatively thick lenses, meaning that for individual with such a condition, glasses frames designed for relatively thin lenses would be a customized care option unavailable to that patient, as such a variation in the combination of products (e.g., thick lenses with unsuitable frames) would not effectively treat a medical condition of the patient. Similarly, variations in combinations of products that are relatively more expensive than a threshold cost are filtered. Thus unsuitable care options are filtered out for that patient based on data such as the first responsive data set (e.g., data regarding the payment arrangement) and the second responsive data set (e.g., data regarding a medical condition of the patient). Stated differently, the first filtered subset of the customized care options may be said to satisfy both the first responsive data set and the second responsive data set.

The gateway processor 34 may transmit at least a portion of the third set of screen display objects (fourth set of SDOs) comprising a first filtered subset of customized care options for display to the first user session of the first remote point of care terminal 31-1, in response to the filtering (block 214). This filtered subset of the third set of SDOs is called a fourth set of SDOs. The gateway processor 34 may receive a third responsive data set from the first user session (block 216). The third responsive data set comprises selections by the user of specific customized care options selected from the first filtered subset of the customized care options. For example, the user may select lens tinting for corrective lenses to be used in bright environments, or may select anti-condensation coatings for corrective lenses to be used in environments favorable to condensation.

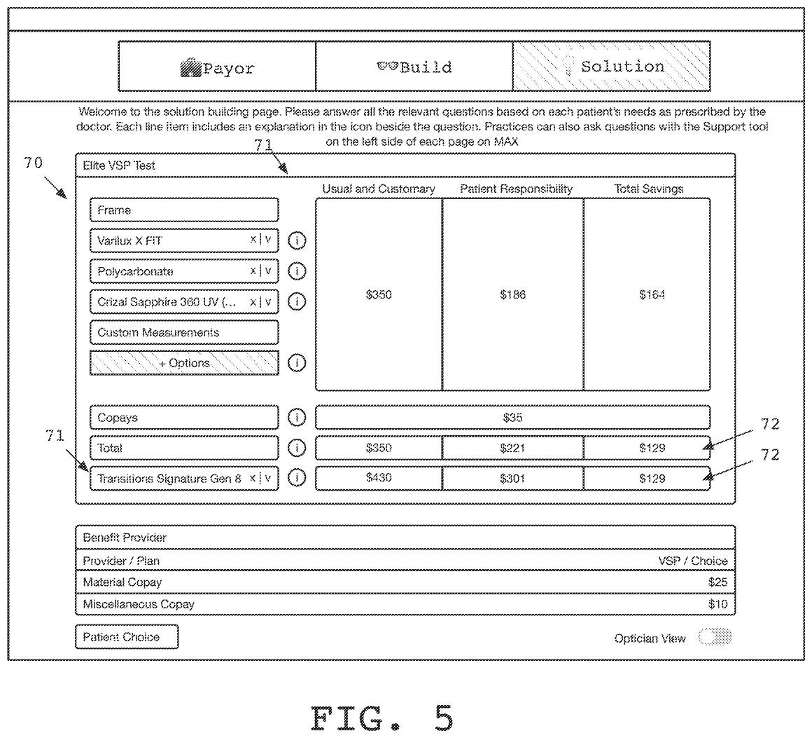

Referring to FIGS. 2A, 2B, and 5, a product build engine 41 may determine at least a subset of treatment packages 71 from a treatment packages database 37, each treatment package of the subset conforming to patient specific care plan information, the patient specific treatment requirements, and the selected customized care options. These treatment packages may be a portion of a fifth set of SDOs. In other words, the product build engine 41 renders a fifth set of SDOs comprising at least a subset of treatment packages from a treatment packages database 37 (block 218). Each SDO of the fifth set of SDOs corresponds to a treatment package that satisfies the various requirements of the specific patient. These requirements are provided in the user's responses to other SDO sets as encapsulated in the first, second, and third responsive data sets. The product build engine 41 evaluates treatment packages from the treatment packages database 37 and determines which treatment packages are medically effective for a patient having the patient specific care plan information, the patient specific treatment requirements, and the selected customized care options. Such treatment packages are the aforementioned subset.

The product build engine 41 may also calculate a price 72 of each treatment package 71 of the subset of treatment packages. In other words, the product build engine 41 calculates a price for each SDO of the fifth set of SDOs (block 220). Such calculation may be based at least partially on the first responsive data set and the second responsive data set and the third responsive data set. For instance, the product build engine 41 may access each data set and retrieve therefrom prices for different products, and then add the prices and subtract discounts (if applicable) to determine a price of each treatment package. Moreover, such price may be modified in response to the payment arrangements of the patient.

The solutions architecture engine 42 may render a sixth set of screen display objects 70 comprising a combination of the fifth set of SDOs and the prices (block 222). Each screen display object of the sixth set may including data representing one of the treatment packages 71 and the price 72 of the one of the treatment packages 71 of the subset of treatment packages.

The gateway processor 34 may transmit the sixth set of screen display objects to the first user session running on the first remote point of care terminal 31-1 (block 224). The gateway processor 34 may receive a fourth responsive data set from the first user session, the fourth responsive data set comprising a user selection of a single screen display object representing a single treatment package of the third set of screen display objects (block 226).

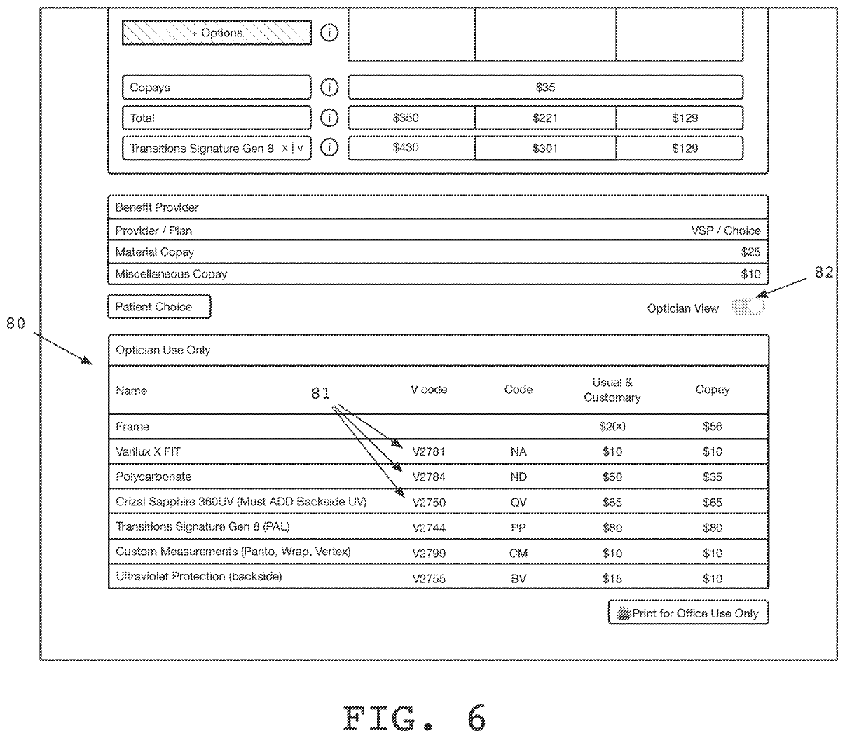

With reference to FIGS. 2A, 2B, and 6, the solutions architecture engine 42 may render a seventh set of screen display objects 80 at least partially retrieved from the products database 38 (block 228). The seventh set 80 may comprise a plurality of order codes 81 associated with component products of the single treatment package that was selected by the user. These order codes may be utilized to order the different component products of the treatment package. Finally, the gateway processor 34 may transmit the fourth set of screen display objects to a second user session running on at least one of the first remote point of care terminal 31-1 and/or a second remote point of care terminal 31-2 (block 230). In this manner, a medical professional, such as an optician, may receive via the second user session, order codes whereby the constituent products of the single treatment package may be ordered.

In various embodiments, the second user session is a portion of the first user session. For example, by operating a button 82 on a screen display, order codes may be revealed within the user session on the same point of care terminal. In further embodiments, the second user session is a different logged in instance of access to the treatment package analytics system 33. For example, a care provider may log in at a later time or different location to retrieve the order codes. Yet furthermore, the second user session may be an automated electronic data connection to a third party system. For instance, the order codes may be automatically transmitted to a supplier so that the associated products are shipped to the care provider.

Accordingly, in some aspects, the present disclosure is directed to an electronic method of treatment package identification and delivery by a treatment package analytics service. For example an electronic method of treatment package identification and delivery by a treatment package analytics service may include rendering, by a payor information engine, a first set of screen display objects at least partially retrieved from a care planning database. The method may include transmitting, by a gateway processor, the first set of screen display objects to a first user session running on a first remote point of care terminal, wherein the first set of screen display objects includes a payor information solicitation.

Additionally, the method may include receiving, by the gateway processor, a first responsive data set from the first user session, the first responsive data set including patient specific care plan information entered in response to the solicitation. Furthermore, the method contemplates rendering, by a payor information engine, a second set of screen display objects at least partially retrieved from a products database and transmitting, by the gateway processor, the second set of screen display objects to the first user session running on the first remote point of care terminal, wherein the second set includes a medical data solicitation. Such a method may also include receiving, by the gateway processor, a second responsive dataset from the first user session, the second responsive data set including patient specific treatment requirements entered in response to the medical data solicitation.

A third set of screen display objects may be filtered by a product build engine. The third set may include a set of customized care options stored in a products database in response to the first responsive data set and the second responsive data set to create a fourth set of screen display objects including a first filtered subset of the customized care options that satisfy both the first responsive data set and the second responsive data set. The method may include transmitting, by the gateway processor, the fourth set of screen display objects including the first filtered subset of customized care options for display to the first user session, in response to the filtering. The method may include receiving, by the gateway processor, a third responsive data set from the first user session, the third responsive data set including selected customized care options selected from the first filtered subset of the customized care options.

The method may also include determining, by the product build engine, at least a subset of treatment packages from a treatment packages database, each treatment package of the subset conforming to patient specific care plan information, the patient specific treatment requirements, and the selected customized care options and rendering, by the product build engine, a fifth set of screen display objects at least partially including the at least the subset of treatment packages from the treatment packages database. Finally, the method may contemplate calculating, by the product build engine, a price of each treatment package of the subset of treatment packages based at least partially on the first responsive data set and the second responsive data set and the third responsive data set.

In various embodiments, the method also includes (i) rendering, by a solutions architecture engine, a sixth set of screen display objects, each screen display object including data representing one of the treatment packages and the price of the one of the treatment packages of the subset of treatment packages, (ii) transmitting, by the gateway processor, the sixth set of screen display objects to the first user session running on the first remote point of care terminal, and (iii) receiving, by the gateway processor, a fourth responsive data set from the first user session, the fourth responsive data set including a user selection of a single screen display object representing a single treatment package of the third set of screen display objects.

In various embodiments, the method includes printing, by the first remote point of care terminal, the seventh set of screen display objects. The method may contemplate rendering, by the solutions architecture engine, a seventh set of screen display objects at least partially retrieved form the products database and including a plurality of order codes associated with components of the single treatment package. In addition to the mentioned rendering, the method may include transmitting, by the gateway processor, the seventh set of screen display objects to a second user session running on the first remote point of care terminal.

In further instances, the method includes rendering, by the solutions architecture engine, a seventh set of screen display objects at least partially retrieved form the products database and including a plurality of order codes associated with components of the single treatment package. In addition to the mentioned rendering, the method may contemplate transmitting, by the gateway processor, the seventh set of screen display objects to a second user session running on a second remote point of care terminal different than the first remote point of care terminal.

In various instances, the method includes rendering, by the solutions architecture engine, a seventh set of screen display objects at least partially retrieved form the products database and including a plurality of order codes associated with components of the single treatment package. Furthermore, the method may include transmitting, by the gateway processor, the seventh set of screen display objects to a third-party vendor automated ordering system.

Non-transient computer readable media may also be provided with data thereon to cause a computer to perform the method. Additionally, systems may be provided configured to perform the method.

As used herein, the term “network,” such as with respect to a network which may comprise at least a portion of network 32 provided in FIG. 2A, includes any cloud, cloud computing system or electronic communications system or method which incorporates hardware and/or software components. Communication among the parties may be accomplished through any suitable communication channels, such as, for example, a telephone network, an extranet, an intranet, Internet, point of interaction device (point of sale device, personal digital assistant (e.g., iPhone®, Palm Pilot®, Blackberry®), cellular phone, kiosk, etc.), online communications, satellite communications, off-line communications, wireless communications, transponder communications, local area network (LAN), wide area network (WAN), virtual private network (VPN), networked or linked devices, keyboard, mouse and/or any suitable communication or data input modality. Moreover, although the system is frequently described herein as being implemented with TCP/IP communications protocols, the system may also be implemented using IPX, Appletalk, IP-6, NetBIOS, OSI, any tunneling protocol (e.g. IPsec, SSH), or any number of existing or future protocols. If the network is in the nature of a public network, such as the Internet, it may be advantageous to presume the network to be insecure and open to eavesdroppers.

A network may be unsecure. Thus, communication over the network may utilize data encryption. Encryption may be performed by way of any of the techniques now available in the art or which may become available—e.g., Twofish, RSA, El Gamal, Schorr signature, DSA, PGP, PKI, GPG (GnuPG), and symmetric and asymmetric cryptosystems.

Any communication, transmission and/or channel discussed herein may include any system or method for delivering content (e.g. data, information, metadata, etc.), and/or the content itself. The content may be presented in any form or medium, and in various embodiments, the content may be delivered electronically and/or capable of being presented electronically. For example, a channel may comprise a website or device (e.g., Facebook, YouTube®, AppleTV®, Pandora®, xBox®, Sony® Playstation®), a uniform resource locator (“URL”), a document (e.g., a Microsoft Word® document, a Microsoft Excel® document, an Adobe .pdf document, etc.), an “ebook,” an “emagazine,” an application or microapplication (as described herein), an SMS or other type of text message, an email, Facebook, twitter, MMS and/or other type of communication technology. In various embodiments, a channel may be hosted or provided by a data partner. In various embodiments, the distribution channel may comprise at least one of a merchant website, a social media website, affiliate or partner websites, an external vendor, a mobile device communication, social media network and/or location based service. Distribution channels may include at least one of a merchant web site, a social media site, affiliate or partner websites, an external vendor, and a mobile device communication. Examples of social media sites include Facebook®, Foursquare®, Twitter®, MySpace®, LinkedIn®, and the like. Examples of affiliate or partner websites include American Express®, Visa®, Google®, and the like. Moreover, examples of mobile device communications include texting, email, and mobile applications for smartphones.

In various embodiments, the methods described herein are implemented using the various particular machines described herein. The methods described herein may be implemented using the below particular machines, and those hereinafter developed, in any suitable combination. Further, as is unambiguous from this disclosure, the methods described herein may result in various transformations of certain articles.

For the sake of brevity, conventional data networking, application development and other functional aspects of the systems (and components of the individual operating components of the systems) may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: client data; merchant data; utility company data; institution data; regulatory agency data; and/or like data useful in the operation of the system. A user computer may include an operating system (e.g., Windows NT®, Windows 95/98/2000®, Windows XP®, Windows Vista®, Windows 7®, OS2, UNIX®, Linux®, Solaris®, MacOS, etc.) as well as various conventional support software and drivers typically associated with computers.

The present system or any part(s) or function(s) thereof may be implemented using hardware, software or a combination thereof and may be implemented in one or more computer systems or other processing systems. However, the manipulations performed by embodiments were often referred to in terms, such as determining or selecting, which are commonly associated with mental operations performed by a human operator. No such capability of a human operator is necessary, possible, or desirable in most cases, in any of the operations described herein. Rather, the operations may be machine operations not performable by mere human activity.

In fact, in various embodiments, the embodiments are directed toward one or more computer systems capable of carrying out the functionality described herein. The computer system includes one or more processors, such as processor. The processor is connected to a communication infrastructure (e.g., a communications bus, cross over bar, or network). Various software embodiments are described in terms of this exemplary computer system. After reading this description, it will become apparent to a person skilled in the relevant art(s) how to implement various embodiments using other computer systems and/or architectures. Computer system can include a display interface that forwards graphics, text, and other data from the communication infrastructure (or from a frame buffer not shown) for display on a display unit.

Computer system also includes a main memory, such as for example random access memory (RAM), and may also include a secondary memory. The secondary memory may include, for example, a hard disk drive and/or a removable storage drive, representing a floppy disk drive, a magnetic tape drive, an optical disk drive, etc. The removable storage drive reads from and/or writes to a removable storage unit in a well-known manner. Removable storage unit represents a floppy disk, magnetic tape, optical disk, etc. which is read by and written to by removable storage drive. As will be appreciated, the removable storage unit includes a computer usable storage medium having stored therein computer software and/or data.

In various embodiments, secondary memory may include other similar devices for allowing computer programs or other instructions to be loaded into computer system. Such devices may include, for example, a removable storage unit and an interface. Examples of such may include a program cartridge and cartridge interface (such as that found in video game devices), a removable memory chip (such as an erasable programmable read only memory (EPROM), or programmable read only memory (PROM)) and associated socket, and other removable storage units and interfaces, which allow software and data to be transferred from the removable storage unit to computer system.

Computer system may also include a communications interface. Communications interface allows software and data to be transferred between computer system and external devices. Examples of communications interface may include a modem, a network interface (such as an Ethernet card), a communications port, a Personal Computer Memory Card International Association (PCMCIA) slot and card, etc. Software and data transferred via communications interface are in the form of signals which may be electronic, electromagnetic, and optical or other signals capable of being received by communications interface. These signals are provided to communications interface via a communications path (e.g., channel). This channel carries signals and may be implemented using wire, cable, fiber optics, a telephone line, a cellular link, a radio frequency (RF) link, wireless and other communications channels.

The terms “computer program medium” and “computer usable medium” and “computer readable medium” are used to generally refer to media such as removable storage drive and a hard disk installed in hard disk drive. These computer program products provide software to computer system.

Computer programs (also referred to as computer control logic) are stored in main memory and/or secondary memory. Computer programs may also be received via communications interface. Such computer programs, when executed, enable the computer system to perform the features as discussed herein. In particular, the computer programs, when executed, enable the processor to perform the features of various embodiments. Accordingly, such computer programs represent controllers of the computer system.

In various embodiments, software may be stored in a computer program product and loaded into computer system using removable storage drive, hard disk drive or communications interface. The control logic (software), when executed by the processor, causes the processor to perform the functions of various embodiments as described herein. In various embodiments, hardware components such as application specific integrated circuits (ASICs) are implemented. Implementation of the hardware state machine so as to perform the functions described herein will be apparent to persons skilled in the relevant art(s).

The various system components may be independently, separately or collectively suitably coupled to the network via data links which includes, for example, a connection to an Internet Service Provider (ISP) over the local loop as is typically used in connection with standard modem communication, cable modem, Dish Networks®, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

“Cloud” or “Cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand. For more information regarding cloud computing, see the NIST's (National Institute of Standards and Technology) definition of cloud computing at http://nvlpubs.nist.gov/nistpubs/Legacy/SP/nistspecialpublication800-145.pdf (last visited April 2021), which is hereby incorporated by reference in its entirety.

As used herein, “transmit” may include sending electronic data from one system component to another over a network connection. Additionally, as used herein, “data” may include encompassing information such as commands, queries, files, data for storage, and the like in digital or any other form.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MySQL, Interbase, etc., may be used to provide an Active Data Object (ADO) compliant database management system. In one embodiment, the Apache web server is used in conjunction with a Linux operating system, a MySQL database, and the Perl, PHP, and/or Python programming languages.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), AJAX (Asynchronous Javascript And XML), helper applications, plug-ins, and the like. A server may include a web service that receives a request from a web server, the request including a URL (http://yahoo.com/stockquotes/ge) and an IP address (123.56.789.234). The web server retrieves the appropriate web pages and sends the data or applications for the web pages to the IP address. Web services are applications that are capable of interacting with other applications over a communications means, such as the internet. Web services are typically based on standards or protocols such as XML, SOAP, AJAX, WSDL and UDDI. Web services methods are well known in the art, and are covered in many standard texts. See, e.g., ALEX NGHIEM, IT WEB SERVICES: A ROADMAP FOR THE ENTERPRISE (2003), hereby incorporated by reference.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, Java, JavaScript, VBScript, Macromedia Cold Fusion, COBOL, Microsoft Active Server Pages, assembly, PERL, PHP, awk, Python, Visual Basic, SQL Stored Procedures, PL/SQL, any UNIX shell script, and extensible markup language (XML) with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

The system may be embodied as a customization of an existing system, an add-on product, a processing apparatus executing upgraded software, a standalone system, a distributed system, a method, a data processing system, a device for data processing, and/or a computer program product. Accordingly, any portion of the system or a module may take the form of a processing apparatus executing code, an internet based embodiment, an entirely hardware embodiment, or an embodiment combining aspects of the internet, software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

These computer program instructions may be loaded onto a programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user windows, webpages, websites, web forms, prompts, etc. Practitioners will appreciate that the illustrated steps described herein may comprise in any number of configurations including the use of windows, webpages, web forms, popup windows, prompts and the like. It should be further appreciated that the multiple steps as illustrated and described may be combined into single webpages and/or windows but have been expanded for the sake of simplicity. In other cases, steps illustrated and described as single process steps may be separated into multiple webpages and/or windows but have been combined for simplicity.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

Systems, methods and computer program products are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Although the disclosure includes a method, it is contemplated that it may be embodied as computer program instructions on a tangible computer-readable carrier, such as a magnetic or optical memory or a magnetic or optical disk. All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

The foregoing method descriptions and the process flow diagrams are provided merely as illustrative examples and are not intended to require or imply that the steps of the various embodiments must be performed in the order presented. The steps in the foregoing embodiments may be performed in any order. Words such as “then,” “next,” etc., are not intended to limit the order of the steps; these words are simply used to guide the reader through the description of the methods. Although process flow diagrams may describe the operations as a sequential process, many of the operations can be performed in parallel or concurrently. In addition, the order of the operations may be rearranged. A process may correspond to a method, a function, a procedure, a subroutine, a subprogram, etc. When a process corresponds to a function, its termination may correspond to a return of the function to the calling function or the main function.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or the like, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory, processor-readable storage media may be any available media that may be accessed by a computer. By way of example, and not limitation, such non-transitory, processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed:

1. A data processing system for a computer memory, comprising:
means for configuring a memory device according to a hybrid decision tree, said hybrid decision tree including:
a plurality of nodes, each node comprising at least one directional associative link to or from another node, each node associated with a function performed on an input variable, wherein each directional associative link is agnostic to a hierarchical layer of the associated nodes in the hybrid decision tree; and
means for executing functions associated with each node of the hybrid decision tree according to the corresponding directional associative links.

2. The data processing system of claim 1, wherein a first node at a first layer of the hybrid decision tree comprises a link to a second node at the first layer of the hybrid decision tree and a third node at a different second layer of the hybrid decision tree.

3. An electronic method of treatment package identification and delivery by a treatment package analytics service, the method comprising:
rendering, by a payor information engine, a first set of screen display objects at least partially retrieved from a care planning database;
transmitting, by a gateway processor, the first set of screen display objects to a first user session running on a first remote point of care terminal, wherein the first set of screen display objects comprises a payor information solicitation;
receiving, by the gateway processor, a first responsive data set from the first user session, the first responsive data set comprising patient specific care plan information entered in response to the solicitation;
rendering, by a payor information engine, a second set of screen display objects at least partially retrieved from a products database;
transmitting, by the gateway processor, the second set of screen display objects to the first user session running on the first remote point of care terminal, wherein the second set comprises a medical data solicitation;
receiving, by the gateway processor, a second responsive dataset from the first user session, the second responsive data set comprising patient specific treatment requirements entered in response to the medical data solicitation;
filtering, by a product build engine according to a hybrid decision tree, a third set of screen display objects comprising a set of customized care options stored in a products database in response to the first responsive data set and the second responsive data set to create a fourth set of screen display objects comprising a first filtered subset of the customized care options that satisfy both the first responsive data set and the second responsive data set,
wherein the hybrid decision tree comprises a plurality of nodes corresponding to the responsive data set and customized care options, each node comprising at least one directional associative link to or from another node, each node associated with a function performed on an input variable, wherein each directional associative link is agnostic to a hierarchical layer of the associated nodes in the hybrid decision tree;
transmitting, by the gateway processor, the fourth set of screen display objects comprising the first filtered subset of customized care options for display to the first user session, in response to the filtering;
receiving, by the gateway processor, a third responsive data set from the first user session, the third responsive data set comprising selected customized care options selected from the first filtered subset of the customized care options;
determining, by the product build engine, at least a subset of treatment packages from a treatment packages database, each treatment package of the subset conforming to patient specific care plan information, the patient specific treatment requirements, and the selected customized care options and rendering, by the product build engine, a fifth set of screen display objects at least partially comprising the at least the subset of treatment packages from the treatment packages database; and
calculating, by the product build engine, a price of each treatment package of the subset of treatment packages based at least partially on the first responsive data set and the second responsive data set and the third responsive data set.

4. The method of claim 3, further comprising:
rendering, by a solutions architecture engine, a sixth set of screen display objects, each screen display object including data representing one of the treatment packages and the price of the one of the treatment packages of the subset of treatment packages;
transmitting, by the gateway processor, the sixth set of screen display objects to the first user session running on the first remote point of care terminal; and
receiving, by the gateway processor, a fourth responsive data set from the first user session, the fourth responsive data set comprising a user selection of a single screen display object representing a single treatment package of the third set of screen display objects.

5. The method of claim 4, further comprising printing, by the first remote point of care terminal, the seventh set of screen display objects.

6. The method of claim 4, further comprising:
rendering, by the solutions architecture engine, a seventh set of screen display objects at least partially retrieved form the products database and comprising a plurality of order codes associated with components of the single treatment package; and
transmitting, by the gateway processor, the seventh set of screen display objects to a second user session running on the first remote point of care terminal.

7. The method of claim 4, further comprising:
rendering, by the solutions architecture engine, a seventh set of screen display objects at least partially retrieved form the products database and comprising a plurality of order codes associated with components of the single treatment package; and
transmitting, by the gateway processor, the seventh set of screen display objects to a second user session running on a second remote point of care terminal different than the first remote point of care terminal.

8. The method of claim 4, further comprising:
rendering, by the solutions architecture engine, a seventh set of screen display objects at least partially retrieved form the products database and comprising a plurality of order codes associated with components of the single treatment package; and
transmitting, by the gateway processor, the seventh set of screen display objects to a third-party vendor automated ordering system.

9. A non-transient computer readable medium containing program instructions for causing at least one computer to perform an electronic method of treatment package identification and delivery by a treatment package analytics service, the method comprising:
rendering, by a payor information engine, a first set of screen display objects at least partially retrieved from a care planning database;
transmitting, by a gateway processor, the first set of screen display objects to a first user session running on a first remote point of care terminal, wherein the first set of screen display objects comprises a payor information solicitation;
receiving, by the gateway processor, a first responsive data set from the first user session, the first responsive data set comprising patient specific care plan information entered in response to the solicitation;
rendering, by a payor information engine, a second set of screen display objects at least partially retrieved from a products database;
transmitting, by the gateway processor, the second set of screen display objects to the first user session running on the first remote point of care terminal, wherein the second set comprises a medical data solicitation;
receiving, by the gateway processor, a second responsive dataset from the first user session, the second responsive data set comprising patient specific treatment requirements entered in response to the medical data solicitation;
filtering, by a product build engine according to a hybrid decision tree, a third set of screen display objects comprising a set of customized care options stored in a products database in response to the first responsive data set and the second responsive data set to create a fourth set of screen display objects comprising a first filtered subset of the customized care options that satisfy both the first responsive data set and the second responsive data set,
wherein the hybrid decision tree comprises a plurality of nodes corresponding to the responsive data set and customized care options, each node comprising at least one directional associative link to or from another node, each node associated with a function performed on an input variable, wherein each directional associative link is agnostic to a hierarchical layer of the associated nodes in the hybrid decision tree;
transmitting, by the gateway processor, the fourth set of screen display objects comprising the first filtered subset of customized care options for display to the first user session, in response to the filtering;
transmitting, by the gateway processor, the fourth set of screen display objects comprising the first filtered subset of customized care options for display to the first user session, in response to the filtering;
receiving, by the gateway processor, a third responsive data set from the first user session, the third responsive data set comprising selected customized care options selected from the first filtered subset of the customized care options;
determining, by the product build engine, at least a subset of treatment packages from a treatment packages database, each treatment package of the subset conforming to patient specific care plan information, the patient specific treatment requirements, and the selected customized care options and rendering, by the product build engine, a fifth set of screen display objects at least partially comprising the at least the subset of treatment packages from the treatment packages database; and
calculating, by the product build engine, a price of each treatment package of the subset of treatment packages based at least partially on the first responsive data set and the second responsive data set and the third responsive data set.

10. The non-transient computer readable medium of claim 9, wherein the method further comprises:
rendering, by a solutions architecture engine, a sixth set of screen display objects, each screen display object including data representing one of the treatment packages and the price of the one of the treatment packages of the subset of treatment packages;
transmitting, by the gateway processor, the sixth set of screen display objects to the first user session running on the first remote point of care terminal; and
receiving, by the gateway processor, a fourth responsive data set from the first user session, the fourth responsive data set comprising a user selection of a single screen display object representing a single treatment package of the third set of screen display objects.

11. The non-transient computer readable medium of claim 10, wherein the method further comprises printing, by the first remote point of care terminal, the seventh set of screen display objects.

12. The non-transient computer readable medium of claim 10, wherein the method further comprises:
rendering, by the solutions architecture engine, a seventh set of screen display objects at least partially retrieved form the products database and comprising a plurality of order codes associated with components of the single treatment package; and
transmitting, by the gateway processor, the seventh set of screen display objects to a second user session running on the first remote point of care terminal.

13. The non-transient computer readable medium of claim 10, wherein the method further comprises:
rendering, by the solutions architecture engine, a seventh set of screen display objects at least partially retrieved form the products database and comprising a plurality of order codes associated with components of the single treatment package; and
transmitting, by the gateway processor, the seventh set of screen display objects to a second user session running on a second remote point of care terminal different than the first remote point of care terminal.

14. The non-transient computer readable medium of claim 10, wherein the method further comprises:
rendering, by the solutions architecture engine, a seventh set of screen display objects at least partially retrieved form the products database and comprising a plurality of order codes associated with components of the single treatment package; and
transmitting, by the gateway processor, the seventh set of screen display objects to a third-party vendor automated ordering system.

15. A system for electronic treatment package identification and delivery by a treatment package analytics service, the system comprising:
a payor information engine connected to a gateway processor, the payor information engine configured render a first set of screen display objects at least partially retrieved from a care planning database;
wherein the gateway processor is configured to:
transmit the first set of screen display objects to a first user session running on a first remote point of care terminal, wherein the first set of screen display objects comprises a payor information solicitation, and
receive a first responsive data set from the first user session, the first responsive data set comprising patient specific care plan information entered in response to the solicitation;
wherein the payor information engine is also configured to render a second set of screen display objects at least partially retrieved from a products database;
wherein the gateway processor is also configured to:
transmit the second set of screen display objects to the first user session running on the first remote point of care terminal, wherein the second set comprises a medical data solicitation, and
receive a second responsive dataset from the first user session, the second responsive data set comprising patient specific treatment requirements entered in response to the medical data solicitation;
a product build engine configured to filter, according to a hybrid decision tree, a third set of screen display objects comprising a set of customized care options stored in a products database in response to the first responsive data set and the second responsive data set to create a fourth set of screen display objects comprising a first filtered subset of the customized care options that satisfy both the first responsive data set and the second responsive data set,
wherein the hybrid decision tree comprises a plurality of nodes corresponding to the responsive data set and customized care options, each node comprising at least one directional associative link to or from another node, each node associated with a function performed on an input variable, wherein each directional associative link is agnostic to a hierarchical layer of the associated nodes in the hybrid decision tree;
wherein the gateway processor is further configured to:
transmit the fourth set of screen display objects comprising the first filtered subset of customized care options for display to the first user session, in response to the filtering, and
receive a third responsive data set from the first user session, the third responsive data set comprising selected customized care options selected from the first filtered subset of the customized care options; and
wherein the product build engine is also configured to:
determine at least a subset of treatment packages from a treatment packages database, each treatment package of the subset conforming to patient specific care plan information, the patient specific treatment requirements, and the selected customized care options and rendering, by the product build engine, a fifth set of screen display objects at least partially comprising the at least the subset of treatment packages from the treatment packages database, and
calculate a price of each treatment package of the subset of treatment packages based at least partially on the first responsive data set and the second responsive data set and the third responsive data set.

16. The system of claim 15, further comprising:
a solutions architecture engine configured to render a sixth set of screen display objects, each screen display object including data representing one of the treatment packages and the price of the one of the treatment packages of the subset of treatment packages;
wherein the gateway processor is further configured to:
transmit the sixth set of screen display objects to the first user session running on the first remote point of care terminal, and
receive a fourth responsive data set from the first user session, the fourth responsive data set comprising a user selection of a single screen display object representing a single treatment package of the third set of screen display objects.

17. The system of claim 16, wherein the first remote point of care terminal is configured to print the seventh set of screen display objects.

18. The system of claim 16,
wherein the solutions architecture engine is configured to render a seventh set of screen display objects at least partially retrieved form the products database and comprising a plurality of order codes associated with components of the single treatment package, and
wherein the gateway processor is configured to transmit the seventh set of screen display objects to a second user session running on the first remote point of care terminal.

19. The system of claim 16,
wherein the solutions architecture engine is configured to render a seventh set of screen display objects at least partially retrieved form the products database and comprising a plurality of order codes associated with components of the single treatment package, and
wherein the gateway processor is configured to transmit the seventh set of screen display objects to a second user session running on a second remote point of care terminal different than the first remote point of care terminal.

20. The system of claim 16,
wherein the solutions architecture engine is configured to render a seventh set of screen display objects at least partially retrieved form the products database and comprising a plurality of order codes associated with components of the single treatment package, and
wherein the gateway processor is configured to transmit the seventh set of screen display objects to a third-party vendor automated ordering system.
